(19) United States
(12) Patent Application Publication
(10) Pub. No.: US 2006/0009639 A1
(43) Pub. Date: Jan. 12, 2006

(54) Process for the preparation of cefpodoxime proxetil

(75) Inventors: Pandurang Balwant Deshpande, Chennai (IN); Gautam Kumar Das, Chennai (IN); Parven Kumar Luthra, Chennai (IN); Pramod Narayan Deshpande, Chennai (IN); Selvraj Subramanian, Chennai (IN); Ramakrishna Kamma, Chennai (IN)

Correspondence Address: OLIFF & BERRIDGE, PLC, P.O. BOX 19928, ALEXANDRIA, VA, 22320, US

(73) Assignee: Orchid Chemicals & Pharmaceuticals Limited, Chennai (IN)

(21) Appl. No.: 10/535,923

(22) PCT Filed: Nov. 19, 2003
(86) PCT No.: PCT/IB03/05310

§ 371 Date: May 23, 2005

(30) Foreign Application Priority Data
Nov. 22, 2002 (IN) 870/MAS/2002

(51) Int. Cl. C07D 501/14

(52) U.S. Cl. 540/228

(57) ABSTRACT

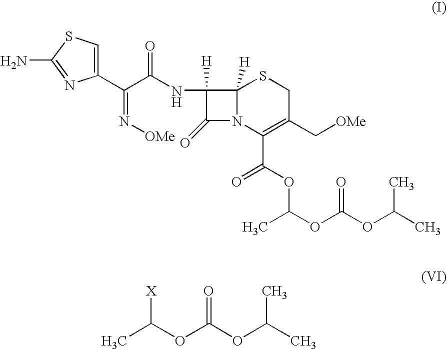

An improved process for the preparation of cefpodoxime proxetil of the formula (I) the said process comprises reacting cefpodoxime acid with 1-haloethyl isopropyl carbonate of the formula (VI) where X represents halogen atom such as chlorine, bromine or iodine using a base such as tetramethylguanidine, di-isopropylethyl amine, 1,5-diazabicyclo(4.3.0)non-5-ene (DBN), 1,4-diazabicyclo[2.2.2]octane (DABCO) in the presence of a solvent at a temperature in the range of −30 ° C. to 30 ° C. to produce cefpodoxime proxetil of the formula (I) and isolating the pure cefpodoxime proxetil of the formula (I).

### FIELD OF THE INVENTION

The present invention relates to an improved process for the preparation of cephalosporin antibiotic. More particularly, the present invention relates to an improved process for the preparation of cefpodoxime proxetil of the formula (I).

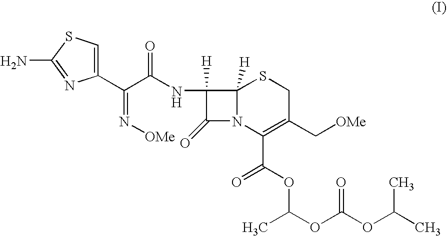

The present invention more particularly provides an improved process for the preparation of cefpodoxime proxetil of the formula (I), which process gives the product with reduced percentage of Δ³ isomer. The Δ³ isomer is named as per the IUPAC nomenclature of position of carboxylic acid as 2-carboxylic acid on the cephem moiety. This isomer can also be called as Δ² isomer as per the conventional nomenclature wherein the position of carboxylic acid is numbered as 4-carboxylic acid on the cephem moiety. However, we choose to use the IUPAC nomenclature and identified the unwanted isomer as Δ³ isomer.

### BACKGROUND OF THE INVENTION

Cefpodoxime proxetil is a third generation cephalosporin antibiotic for oral administration and has a broader antibacterial spectrum over the general gram positive and gram negative bacteria, especially against Streptococci than other antibiotics for oral administration. The activity is primarily due to the cefpodoxime acid which is generated easily in vivo by the hydrolysis of the proxetil.

U.S. Pat. No. 4,486,425 describes a process for the preparation of cefpodoxime proxetil. The process comprises reacting cefpodoxime acid of the formula (II) 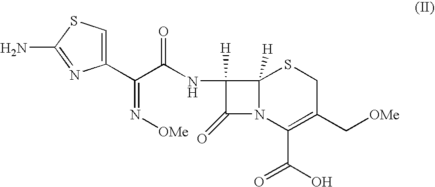
with 1-iodoethyl isopropyl carbonate in the presence of a base and solvent.

The bases used are potassium carbonate, sodium carbonate, sodium bicarbonate, triethylamine, dicyclohexylamine, pyridine or N,N-dimethylaniline. The reaction mixture is treated with water-immiscible solvent, washed successively with an aqueous solution of potassium bisulphate and an aqueous basic solution and then dried, after which the solvent is distilled off to give the desired product. The compound is further purified by chromatographic techniques.

International publication number WO02/68429 claims a process for the preparation of cefpodoxime proxetil and its purification. The process comprises reacting cefpodoxime acid of the formula (II) 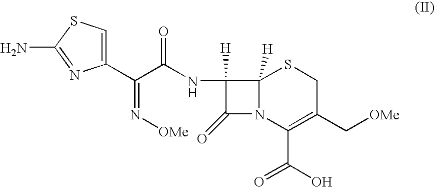
with 1-iodoethyl isopropyl carbonate optionally in the presence of a base. The bases used are DBU, sodium carbonate and the like. The purification is carried out using a two step methodology.

In our U.S. Pat. No. 6,388,070 we disclosed a process for preparing a compound of formula (V), wherein, R₁ represents H, trityl, methyl etc., R₂ represents H, phenyl, etc., R₄ is CH₃, CH═CH, etc., R₅ is H or a salt or a carboxylic protecting group; R₆ is H or trimethylsilyl; comprising acylating the compound of formula (III) with compound of formula (IV) in the presence of an organic solvent, organic base and a silylating agent at a temperature in the range of −10° C. to +30° C. The reaction is shown in scheme I below: 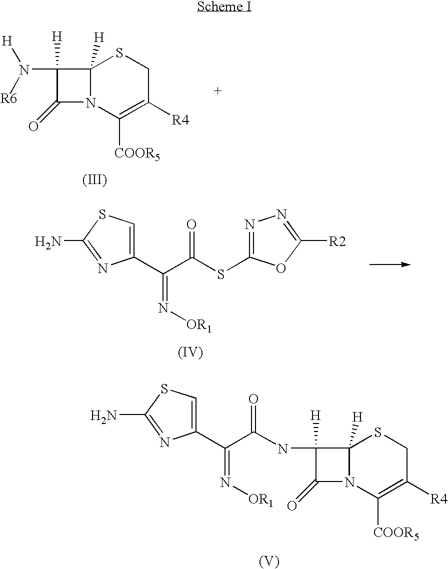
The above process though broadly claims the process for the preparation of the esters does not specifically envisage how the ester compounds such as cefpodoxime proxetil are prepared from the cefpodoxime acid.

The conventional processes reported as of date produces a product with Δ³ isomer in the range of 1.5 to 2.0. There is a possibility of formation of a is high molecular weight impurities in the process. Such high molecular weight impurities percentage has also been reduced by the process of the present invention.

Considering the foregoing limitations, we undertook an investigation in our lab to identify a process, which yields a product with high quality and with less number of impurities. This would permit commercializing the production of highly pure cefpodoxime proxetil without any further purifications, which in turn reduces the consumption of the solvents for purification, which directly has effect on the total cost of the process and is environmental friendly.

### OBJECTIVES OF THE INVENTION

The main objective of the present invention is to provide an improved commercially viable process for the preparation of cefpodoxime proxetil of the formula (I), which would be easy to implement in manufacturing scale.

Another objective of the present invention is to provide an improved commercially viable process for the preparation of cefpodoxime proxetil of the formula (a), which has reduced percentages of impurities in the final product.

Another objective of the present invention is to provide an improved commercially viable process for the preparation of cefpodoxime proxetil of the formula (I), without any purification steps, thereby reducing the solvent usage.

Another objective is to provide an improved commercially viable process for the preparation of cefpodoxime proxetil of the formula (I), which avoids the use of chromatography for purification.

### SUMMARY OF THE INVENTION

Accordingly, the present invention provides an improved process for the preparation of cefpodoxime proxetil of the formula (I) 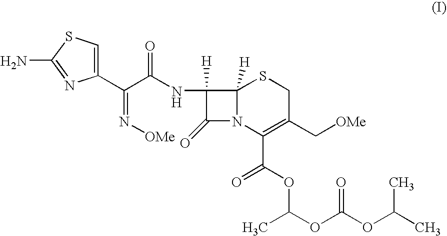
the said process comprising the steps of:
i) reacting cefpodoxime acid of the formula (II) with 1-haloethyl isopropyl carbonate of the formula (VI) where X represents halogen atom such as chlorine, bromine or iodine using a base selected from tetrarnethylguanidine, di-isopropylethyl amine, 1,5-diazabicyclo(4.3.0)non-5-ene (DBN) or 1,4-diazabicyclo[2.2.2]octane (DABCO) in the presence of a solvent at a temperature in the range of −30° C. to 30° C. to produce cefpodoxime proxetil of the formula (I) and
ii) isolating the pure cefpodoxime proxetil of the formula (I).
   The reaction is shown in scheme-II below:

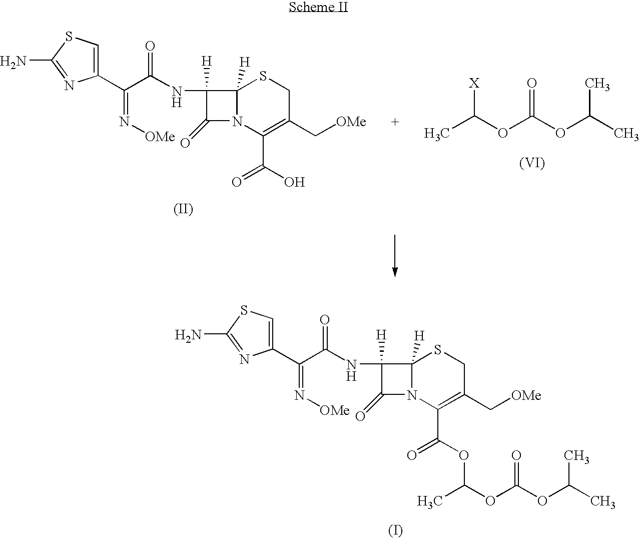

### DETAILED DESCRIPTION OF THE INVENTION

In yet another embodiment of the present invention, the solvent used in step (i) is selected from TlF, dichloromethane, acetone, butan-2-one, acetonitrile, DMAc, DMF, DMSO, or a mixture thereof, preferably DMAc, DMF.

In yet another embodiment of the present invention, the compound of formula (I) obtained is a syn isomer.

In another embodiment of the present invention, the isolation in step (ii) is carried out using water miscible solvent and water or an acid followed by basification. The water miscible solvent is selected from methanol, ethanol, iso-propanol and the like. The acid used is selected from hydrochloric acid, sulfuric acid, and the like. The basification is carried out using base such as ammonia, triethyl amine and the like.

In another embodiment of the present invention, there is provided a process for the preparation of pure cefpodoxime proxteil comprising purifying the cefpodoxime proxetil using water miscible solvent and water or an acid. The water miscible solvent is selected from methanol, ethanol, iso-propanol and the like. The acid used is selected from hydrochloric acid, sulfuric acid, and the like.

The foregoing technique has been found to be markedly attractive, both from commercial point of view, as well as from manufacturing viewpoint, and affords good quality of cefpodoxime proxetil of the formula (I) for the following reasons:
1. The use of the bases mentioned above for the reaction yields a product with reduced percentage of Δ³ isomer.
2. Less number of solvents are used for the purification of the crude product.
3. High molecular weight impurities are reduced by the treatment of the crude product solution with acid.

It is reported in the International publication number WO 01/34611 that the commercial cefpodoxime proxetil contains 1.44% of the Δ³ isomer and the product produced by the process of the present invention contains around 0.3 to 0.6% of the Δ³ isomer.

Many other beneficial results can be obtained by applying disclosed invention in a different manner or by modifying the invention with the scope of disclosure.

The present invention is illustrated with the following examples, which should not be construed as limiting to the scope of the invention.

The cefpodoxime free acid used for the preparation of cefpodoxime proxetil of the present invention is prepared by any method disclosed in the prior art.

### EXAMPLE 1

### Preparation of [6R-[6α, 7β(Z))]]-7-[[(2-Amino-4-thiazolyl)(methoxyimino) acetyl]amino]-3-(methoxy-methyl)-8-oxo-5-thia-1-azabicyclo[4.2.0]oct-2-ene-2- carboxylic acid 1-[[(1-methylethoxy)carbonyl]oxy]ethyl ester (Cefpodoxime proxetil)

1,1,3,3-Tetramethylguanidine (26.8 g) was added to a mixture of [6R-[6α,7β(Z))]]-7-[[(2-amino-4-thiazolyl)(methoxyimino)acetyl]amino]-3-(methoxy-methyl)-8-oxo-5-thia-1-azabicyclo[4.2.0]oct-2-ene-2-carboxylic acid (Cefpodoxime acid, 100 g) in dimethyl acetamide (500 ml) at −10 to −12° C., and stirring was continued for 30 minutes at −4 to −6° C. for 30 minutes. The reaction mixture was cooled to −15° C. and 1-iodoethylisopropylcarbonate (57.4 g, 100% purity basis) was added. Stirring was continued for 30-90 minutes at −8 to −10° C. After completion of reaction, the reaction mixture was added into a mixture of water (4000 ml) and ethyl acetate (1200 ml). Sodium thio sulphate (25 g) and EDTA (1.0 g) were added. The mixture was stirred for 15 minutes at 30-32° C. and the layers were separated, the lower aqueous layer was washed with ethyl acetate (100 ml). The two organic layers were washed subsequently thrice with solution of sodium chloride (20 g) in water (400 ml). The organic layer was stirred with activated carbon (15 g) for 30 minutes and filtered through a bed of Hyflo Supercel, the bed being washed with ethyl acetate (100 ml). The filtrate and wash were combined and evaporated under vacuum until thick residue. Methyl alcohol (500 ml) was added and stirred for 15 minutes and evaporated under vacuum at 25-35° C. until thick residue. Methyl alcohol (400 ml) and 17-18% w/w aqueous hydrochloric acid (40 ml) were added and stirring was continued for 15 minutes at 30° C. The resultant solution was added in water (2000 ml) for 30-45 minutes at 30 to 32° C. by maintaining the pH 3.5 to 4.0, by addition of 4% aqueous ammonia solution (45 ml) to complete the crystallization. The product slurry was cooled to 5° C. and filtered, the cake was washed with water (500 ml), sucked dry and dried under vacuum at 45-50° C. for 8-10 hours to give Cefpodoxime Proxetil (yield 115-120 g; purity 99.22% and Δ³ isomer is 0.36%).

### EXAMPLE 2

### Preparation of [6R-[6α,7β(Z))]]-7-[[(2-Amino-4-thiazolyl)(methoxyimino)acetyl]amino]-3-(methoxy-methyl)-8-oxo-5-thia-1-azabicyclo[4.2.0]oct-2-ene-2-carboxylic acid 1-[[(1-methylethoxy)carbonyl]oxy]ethyl ester (Cefpodoxime proxetil)

Di-isopropyl ethylamine (34.8 g) was added to a mixture of [6R-[6α,7β(Z))]]-7-[[(2-amino-4-thiazolyl)(methoxyimino)acetyl]amino]-3-(methoxy-methyl)-8-oxo-5-thia-1-azabicyclo[4.2.0]oct-2-ene-2-carboxylic acid (Cefpodoxime acid, 100 g) in dimethyl acetamide (500 ml) at −10 to −12° C., and stirring was continued for 30 minutes at −4 to −6° C. for 30 minutes. The reaction mixture was cooled to −15° C. and 1-iodoethylisopropylcarbonate (57.4 g, 100% purity basis) was added. Stirring was continued for 30-90 minutes at −8 to −10° C. After completion of reaction, the reaction mixture was added into a mixture of water (4000 ml) and ethyl acetate (1200 ml). Sodium thio sulphate (25 g) and EDTA (1.0 g) were added. The mixture was stirred for 15 minutes at 30-32° C. and the layers were separated, the lower aqueous layer was washed with ethyl acetate (100 ml). The two organic layers were washed subsequently thrice with solution of sodium chloride (20 g) in water (400 ml). The organic layer was stirred with activated carbon (15 g) for 30 minutes and filtered through a bed of Hyflo Supercel, the bed being washed with ethyl acetate (100 ml). The filtrate and wash were combined and evaporated under vacuum until thick residue. Methyl alcohol (500 ml) was added and stirred for 15 minutes and evaporated under vacuum at 25-35° C. until thick residue. Methyl alcohol (400 ml) and 17-18% w/w aqueous hydrochloric acid (40 ml) were added and stirring was continued for 15 minutes at 30° C. The resultant solution was added in water (2000 ml) for 30-45 minutes at 30 to 32° C. by maintaining the pH 3.5 to 4.0, by addition of 4% aqueous ammonia solution (45 ml) to complete the crystallization. The product slurry was cooled to 5° C. and filtered, the cake was washed with water (500 ml), sucked dry and dried under vacuum at 45-50° C. for 8-10 hours to give Cefpodoxime Proxetil (yield 105-110 g; purity 99.14% and Δ³ isomer is 0.55%).

1. An improved process for the preparation of cefpodoxime proxetil of the formula (I) 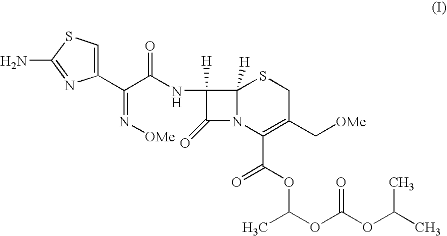
the said process comprising the steps of:
i) reacting cefpodoxime acid of the formula (II) 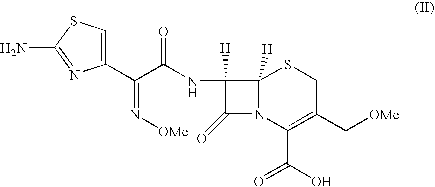
with 1-haloethyl isopropyl carbonate of the formula (VI 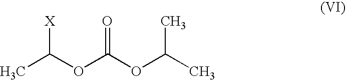
where X represents halogen atom such as chlorine, bromine or iodine using a base selected from tetramethylguanidine, di-isopropylethyl amine, 1,5-diazabicyclo(4.3.0)non-5-ene (DBN) or 1,4-diazabicyclo[2.2.2]octane (DABCO) in the presence of a solvent at a temperature in the range of −30° C. to 30° C. to produce cefpodoxime proxetil of the formula (I) and
ii) isolating the pure cefpodoxime proxetil of the formula (I).

2. The process as claimed in claim 1, wherein the solvent used in step (i) is selected from THF, dichloromethane, acetone, butan-2-one, acetonitrile, DMAc, DMF, DMSO, or a mixture thereof.

3. The process as claimed in claim 1, wherein the isolation in step (ii) is carried out by using water miscible solvent and water or an acid followed by basification.

4. The process as claimed in claim 3, wherein the water miscible solvent is selected from methanol, ethanol or iso-propanol.

5. The process as claimed in claim 3, wherein the acid is selected from hydrochloric acid or sulfuric acid.

6. The process as claimed in claim 3, wherein the base is selected from ammonia or triethyl amine.

7. The process as claimed in claim i, wherein the compound of formula (I) obtained with reduced high molecular weight impurities.

8. The process as claimed in claim 1, wherein the compound of formula (I) obtained is a syn isomer.

9. The process as claimed in claim 1, wherein the compound of formula (I) obtained contains less percentage of Δ³ isomer.

10. An improved process for the preparation of pure cefpodoxime proxteil of formula (I) comprising purifiing the cefpodoxime proxetil using water miscible solvent and water or an acid followed by basification.

11. The process as claimed in claim 10, wherein the water miscible solvent is selected from methanol, ethanol or iso-propanol.

12. The process as claimed in claim 10, wherein the acid is selected from hydrochloric acid or sulfuric acid.

13. The process as claimed in claim 10, wherein the base is selected from ammonia or triethyl amine.

14. The process as claimed in claim 10, wherein the compound of formula (I) obtained with reduced high molecular weight impurities.
